(19) United States
(12) Patent Application Publication
(10) Pub. No.: US 2011/0152665 A1
(43) Pub. Date: Jun. 23, 2011

(54) METHOD AND APPARATUS FOR DESIGNING MRI GRADIENT PULSE WAVEFORM

(76) Inventor: Yongchuan Lai, Beijing (CN)

(21) Appl. No.: 12/976,398

(22) Filed: Dec. 22, 2010

(30) Foreign Application Priority Data
Dec. 23, 2009 (CN) 200910216817.1

(51) Int. Cl. A61B 5/055

(52) U.S. Cl. 600/410

(57) ABSTRACT

A method for designing MRI gradient pulse waveform provided by the present invention firstly defines target peripheral nerve stimulation (PNS) curve; then calculates a gradient pulse waveform by using a relation function between the gradient pulse waveform and PNS value curve based on the target PNS curve.

### CROSS REFERENCE TO RELATED APPLICATIONS

This application claims the benefit of Chinese Patent Application No. 200910216817.1 filed Dec. 23, 2009, which is hereby incorporated by reference in its entirety.

### BACKGROUND OF THE INVENTION

The present invention relates to the technical field of the Magnetic Resonance Imaging (MRI for short), and particularly relates to a method and apparatus for designing MRI gradient pulse waveform in magnetic resonance imaging.

Magnetic resonance imaging requires that an imaged object is positioned in a static magnetic field, neutrons are excited to rotate in the imaged object and sends a detection signal during the rotating process, the detection signal may exist in spatial, three-dimensional modes by using phase encoding of gradient magnetic field and excited magnetic field. A gradient magnetic field system is one of the cores of a MRI system, which utilizes gradient field coil to generate a spatially changing magnetic field weaker than a main magnetic field, the magnetic field changing with spatial position is superimposed on the main magnetic field. It is required that the formed gradient field possesses the following within the imaging range: good linear features; slew time, that is, the time that the gradient field needs to rise from zero to a predetermined stable value, namely the response time, is short, the length of the response time will limit the least available echo time of the imaging system; little power loss, the establishment of the gradient field has to drive all high-power elements in the power circuit to generate powerful current, the heat of the high-power elements has to be dissipated, thus under the condition where a predetermined gradient field intensity is achieved, the power loss of the power source has to be as little as possible; eddy current effect of the lowest degree, the impact of the eddy current effect has to be decreased as far as possible in that the eddy current effect may cause image distortion.

Improvement of the gradient coil performance is very important to magnetic resonance ultrafast imaging, we can say that it is impossible for ultrafast sequence to exist without improvement of the gradient coil. A high gradient field and a high slew rate not only can shorten echo spacing and accelerate signal collection, but also is beneficial to increasing the signal to noise ratio of an image, thus we can say that the development of fast or ultrafast imaging technology in the recent years directly benefits from the improvement of the performance of gradient coil and gradient system. At present, the gradient magnetic field intensity of a 1.5 T superconducting magnetic resonance apparatus equipped with a single gradient amplifier may reach as high as 50 mT/m, and generally may be above 25 mT/m; the gradient slew rate may be as high as 200 mT/m.s, and generally may be above 120 mT/m.s. The gradient magnetic field intensity of a 1.5 T superconducting magnetic resonance apparatus equipped with dual gradient amplifiers may reach as high as 66 mT/m, and the gradient slew rate may reach as high as 200 mT/m.s.

Of course, since rapid change of the gradient magnetic field will have certain impact on human body, and especially will induce peripheral nerve stimulation (PNS for short), a higher gradient field intensity and a higher slew rate are not necessarily better, instead, certain limits are placed to them. Due to such human body safety restrictions, improved hardware capabilities do not always translate to best pulse sequence performance (in terms of less echo time, echo separation, and less repetition time). For instance, Extreme Resonance Module (XRMB) sub-system is capable of providing faster slew rate (200 mT/m/s) and higher amplitude (5G/cm). However, due to the limits of PNS, the actual slew rate is only 117 T/m/s in some applications. Thus various methods for optimizing design of gradient pulse waveform appear, e.g., a patent with U.S. Pat. No. 7,301,34, which provides a method for designing magnetic resonance waveform. However, the method disclosed by the patent is comparatively complicated.

### BRIEF DESCRIPTION OF THE INVENTION

One objective of the present invention is to provide a method for designing gradient pulse waveform, which solves the abovementioned problem, and is designed according to requirement of users to meet the requirement.

Another objective of the present invention is to provide an apparatus for designing gradient pulse waveform, which solves the above-mentioned problem, and is designed according to requirement of users to meet the requirement.

Another objective of the present invention is to provide a method for designing gradient pulse waveform, which solves the above-mentioned problem, and is designed to meet the requirement by sufficiently using the highest slew rate of the system.

Another objective of the present invention is to provide an apparatus for designing gradient pulse waveform, which solves the above-mentioned problem, and is designed to meet the requirement by sufficiently using the highest slew rate of the system.

The method for designing MRI gradient pulse waveform provided by the present invention firstly defines target peripheral nerve stimulation (PNS) curve; then calculates a gradient pulse waveform by using a relation function between the gradient pulse waveform and PNS value curve based on the target PNS curve;

$${R}_{x} ( t ) = \frac{1}{rb} {\int }_{0}^{t} \frac{{\dot{B}}_{x} ( θ ) c}{{( c + t - θ )}^{2}} d θ$$

wherein rb is rheobase, c is chronaxie time, rb and c are both provided by the system; Rx represents PNS value, θ is an intermediate variant from 0˜t; Bₓ represents gradient field intensity, i.e. amplitude; \dot{B}ₓ(θ) represents slew rate, t represents time.

In some embodiments, defining the target PNS curve comprises the following steps: 1) acquiring a slew rate that may be provided by a magnetic resonance system; 2) defining target peripheral nerve stimulation (PNS) curve based on the acquired slew rate, said target PNS curve meets the following requirements: a) reaching a maximal PNS value from the start point with the acquired slew rate, determining the second point of the PNS curve, the time of the second point of the PNS curve being undetermined; b) setting a time randomly, from the second point of the PNS curve as a start, keeping PNS value within a client-set range in the period of time, determining the third point of the PNS curve; c) controlling PNS to decrease from the client-set range to a negative maximal point from the third point with the acquired slew rate, determining the fourth point of the PNS curve, the time of the fourth point of the PNS curve being undetermined; d) keeping PNS value within a client-set range from the fourth point of the PNS curve to the fifth point of the PNS curve, the time of the fifth point of the PNS curve being undetermined.

In some embodiments, the gradient pulse waveform is calculated by: 3) setting the start point of the gradient pulse waveform to be designed; 4) based on the acquired slew rate and the PNS value of the second point of the PNS curve, calculating the time and the amplitude of the second point of the gradient pulse waveform by using the relation function between the gradient pulse waveform and the PNS value curve, the time of the second point of the gradient pulse waveform being consistent with the time of the second point of the PNS curve, whereby determining the second point of the gradient pulse waveform; 5) based on the time and the PNS value of the third point of the PNS curve, calculating the amplitude of the third point of the gradient pulse waveform by using the relation function between the gradient pulse waveform and the PNS value curve, the time of the third point of the gradient pulse waveform being consistent with the time of the third point of the PNS curve, whereby determining the third point of the gradient pulse waveform; 6) based on the acquired slew rate and the PNS value of the fourth point of the PNS curve, calculating the amplitude and the time of the fourth point of the gradient pulse waveform by using the relation function between the gradient pulse waveform and the PNS value curve, the time of the fourth point of the gradient pulse waveform being consistent with the time of the fourth point of the PNS curve, whereby determining the fourth point of the gradient pulse waveform; 7) based on the amplitude of the fifth point of the gradient pulse waveform provided by client or system, and based on the PNS value of the fifth point of the PNS curve, calculating the time of the fifth point of the gradient pulse waveform by using the relation function between the gradient pulse waveform and the PNS value curve, whereby determining the fifth point of the gradient pulse waveform.

In some embodiments, the method for designing MRI gradient pulse waveform, further comprises step 8): calculating the area of the gradient pulse waveform based on the first point to the fifth point of the gradient pulse waveform.

In some embodiments, the method for designing MRI gradient pulse waveform, further comprises the following step: judging whether the area of the gradient pulse waveform area conforms to the target gradient pulse waveform area; if not, adjusting the time of the third point of the PNS curve, the larger the time of the third point of the PNS curve is, the larger the gradient pulse waveform area is; if the calculated area is less than the target area, then increasing the time of the third point of the PNS curve, otherwise, reducing the time of the third point of the PNS curve; repeating steps 4) to 8) until the calculated area conforms to the target gradient pulse waveform area.

In some embodiments, when the time and PNS value of the start point of said target PNS curve are zero, said acquired slew rate is the maximal slew rate of system.

In some embodiments, and with the second point of the PNS curve as a start, the PNS value is kept at a positive maximal value within the period of time; the PNS value is kept at a negative maximal value from the fourth point of the PNS curve to the fifth point of the PNS curve.

In some embodiments, and when the time and the amplitude of the start point of said gradient pulse waveform are zero, and the amplitude of the fifth point of the gradient pulse waveform is zero.

In some embodiments, the time of the fifth point of said gradient pulse waveform is consistent with the time of the fifth point of the PNS curve.

The apparatus for designing MRI gradient pulse waveform provided by the present invention, comprising: a PNS curve defining unit is configured to define target peripheral nerve stimulation (PNS) curve; and a gradient pulse waveform calculating unit is configured to determine a gradient pulse waveform to be designed through calculation; wherein said gradient pulse waveform calculating unit is configured to calculate the gradient pulse waveform by using a relation function between the gradient pulse waveform and the PNS value curve based on the target PNS curve;

$${R}_{x} ( t ) = \frac{1}{rb} {\int }_{0}^{t} \frac{{\dot{B}}_{x} ( θ ) c}{{( c + t - θ )}^{2}} d θ$$

wherein rb is rheobase, c is chronaxie time, rb and c are both provided by the system; Rx represents PNS value, θ is an intermediate variant from 0˜t; Bₓ represents gradient field intensity, i.e. amplitude; \dot{B}ₓ(θ) represents slew rate, t represents time.

In some embodiments, the apparatus for designing MRI gradient pulse waveform, further comprises a slew rate acquiring unit is configured to acquire a slew rate that may be provided by a magnetic resonance system; and the PNS curve defined by said PNS curve defining unit satisfies the following conditions: a) reaching a maximal PNS value from the start point with the acquired slew rate, determining the second point of the PNS curve, the time of the second point of the PNS curve being undetermined; b) setting a time randomly, from the second point of the PNS curve as a start, keeping PNS value within a client-set range in the period of time, determining the third point of the PNS curve; c) controlling PNS to decrease from the client-set range to a negative maximal point from the third point with the acquired slew rate, determining the fourth point of the PNS curve, the time of the fourth point of the PNS curve being undetermined; d) keeping PNS value within a client-set range from the fourth point of the PNS curve to the fifth point of the PNS curve, the time of the fifth point of the PNS curve being undetermined.

In some embodiments, the apparatus for designing MRI gradient pulse waveform, further comprises a setting unit is configured to set the start point of the gradient pulse waveform to be designed; said gradient pulse waveform calculating unit is configured to determine the second point, the third point, the fourth point and the fifth point of the gradient pulse waveform to be designed through calculation.

In some embodiments, the gradient pulse waveform calculating unit is configured to calculate the second point of the gradient pulse waveform by the following way: based on the acquired slew rate and the PNS value of the second point of the PNS curve, calculating the amplitude and the time of the second point of the gradient pulse waveform by using the relation function between the gradient pulse waveform and the PNS value curve, the time of the second point of the gradient pulse waveform being consistent with the time of the second point of the PNS curve, determining the second point of the gradient pulse waveform based on the time and the amplitude; said gradient pulse waveform calculating unit is configured to calculate the third point of the gradient pulse waveform by the following way: based on the time and the PNS value of the third point of the PNS curve, calculating the amplitude of the third point of the gradient pulse waveform by using the relation function between the gradient pulse waveform and the PNS value curve, the time of the third point of the gradient pulse waveform being consistent with the time of the third point of the PNS curve, determining the third point of the gradient pulse waveform based on the time and the amplitude; said gradient pulse waveform calculating unit is configured to calculate the fourth point of the gradient pulse waveform by the following way: based on the acquired slew rate and the PNS value of the fourth point of the PNS curve, calculating the amplitude and the time of the fourth point of the gradient pulse waveform by using the relation function between said gradient pulse waveform and the PNS value curve, the time of the fourth point of the gradient pulse waveform being consistent with the time of the fourth point of the PNS curve, determining the fourth point of the gradient pulse waveform based on the time and the amplitude; said gradient pulse waveform calculating unit is configured to calculate the fifth point of the gradient pulse waveform by the following way: based on the amplitude of the fifth point of the gradient pulse waveform set by client or system, and based on the PNS value of the fifth point of the PNS curve, calculating the time of the fifth point of the gradient pulse waveform by using the relation function between the gradient pulse waveform and the PNS value curve, determining the fifth point of the gradient pulse waveform based on the time and the amplitude, the time of the fifth point of said gradient pulse waveform being consistent with the time of the fifth point of the PNS curve.

In some embodiments, the apparatus for designing MRI gradient pulse waveform further comprises: a gradient pulse waveform area calculating unit is configured to calculate the gradient pulse waveform area based on the first point to the fifth point of the gradient pulse waveform.

In some embodiments, the apparatus for designing MRI gradient pulse waveform, further comprises: a judging unit is configured to judge whether the gradient pulse waveform area conforms to the target gradient pulse waveform area based on the area calculated by the gradient pulse waveform area calculating unit; if not, feeding back information to the PNS curve defining unit; the PNS curve defining unit adjusting the time of the third point of the PNS curve based on the feedback of the judging unit, the larger the time of the third point is, the larger the gradient pulse waveform area is; if the calculated area is less than the target area, then increasing the time of the third point, otherwise, reducing the time of the third point; until the calculated area conforms to the target gradient pulse waveform area.

In some embodiments, and when the time and the PNS value of the start point of said target PNS curve are zero, said acquired slew rate is the maximal slew rate of system.

In some embodiments, the PNS value is kept at a positive maximal value from the second point of the PNS curve as a start; the PNS value is kept at a negative maximal value from the fourth point of the PNS curve to the fifth point of the PNS curve.

In some embodiments, and when the time and the amplitude of the start point of said gradient pulse waveform are zero; the amplitude of the fifth point of said gradient pulse waveform is zero.

Under the premise where the maximal PNS value prescribed by Laws and Regulations is satisfied, the present invention sets target PNS curve, and then designs a gradient pulse waveform by changing slew rate dynamically based on the target PNS curve, whereby the gradient pulse waveform duration may be decreased by sufficiently using the maximal slew rate provided by the system. The gradient pulse designed in such method may improve performance of many fast imaging applications at present, for example, lesser echo time and lesser repetition time.

### BRIEF DESCRIPTION OF THE DRAWINGS

FIGS. 1A and 1B are flow diagrams of a method in an embodiment of the present invention.

FIG. 2 is a schematic diagram of a PNS curve defined in an embodiment of the present invention.

FIG. 3 is a schematic diagram of a gradient pulse waveform designed in an embodiment of the present invention.

FIG. 4 is a schematic diagram of a gradient pulse waveform designed through different numbers of corner points.

FIG. 5 is a schematic diagram of a gradient pulse waveform designed by the prior art under condition equal to that of the implementation of the present invention.

FIGS. 6-8 are schematic diagrams of a gradient pulse waveform designed by the prior art.

FIGS. 9-11 are schematic diagrams of a gradient pulse waveform designed by the present invention under condition equal to that of FIGS. 6-8.

FIG. 12 is a schematic diagram of functional modules of an apparatus for designing gradient pulse waveform in an embodiment of the present invention.

### DETAILED DESCRIPTION OF THE INVENTION

Embodiments of the present invention are illustrated with reference to the figures in details below. The present invention is not limited to the embodiments.

A method for designing MRI gradient pulse waveform provided by the present invention, it firstly defines target peripheral nerve stimulation (PNS) curve; then calculates a gradient pulse waveform by using relation function (1) between the gradient pulse waveform and PNS value curve based on the target curve;

$${R}_{x} ( t ) = \frac{1}{rb} {\int }_{0}^{t} \frac{{\dot{B}}_{x} ( θ ) c}{{( c + t - θ )}^{2}} d θ \qquad function ( 1 )$$

wherein rb is rheobase, c is chronaxie time, rb and c are both provided by system; Rx represents PNS value, θ is an intermediate variant from 0˜t; Bₓ represents gradient field intensity, i.e. amplitude; \dot{B}ₓ(θ) represents slew rate, t represents time.

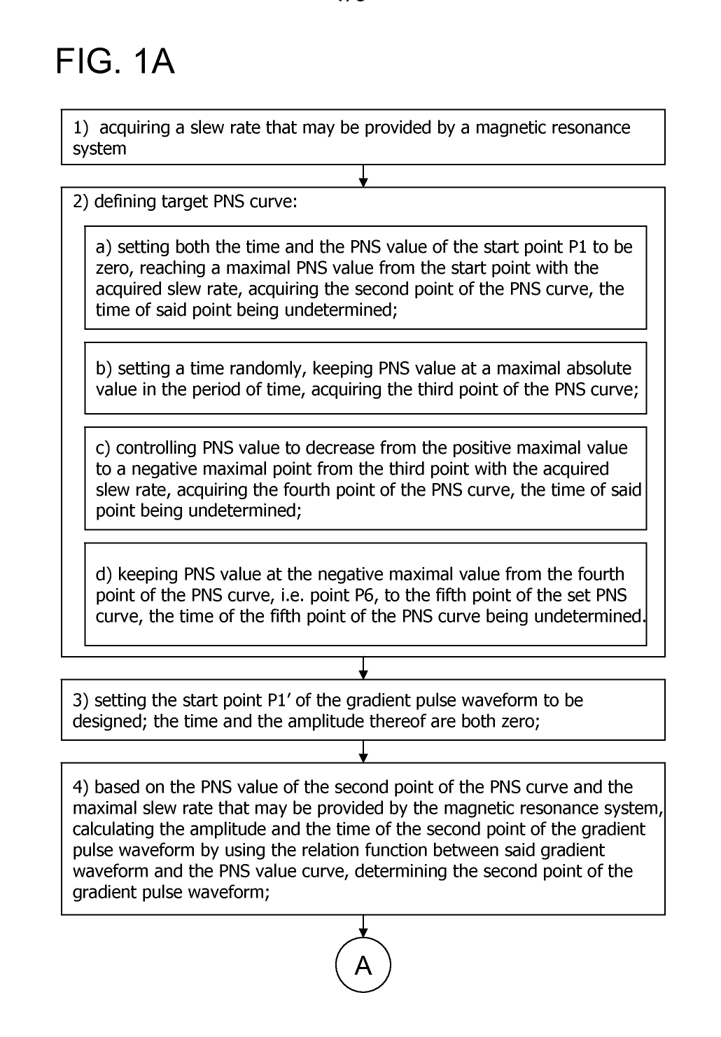

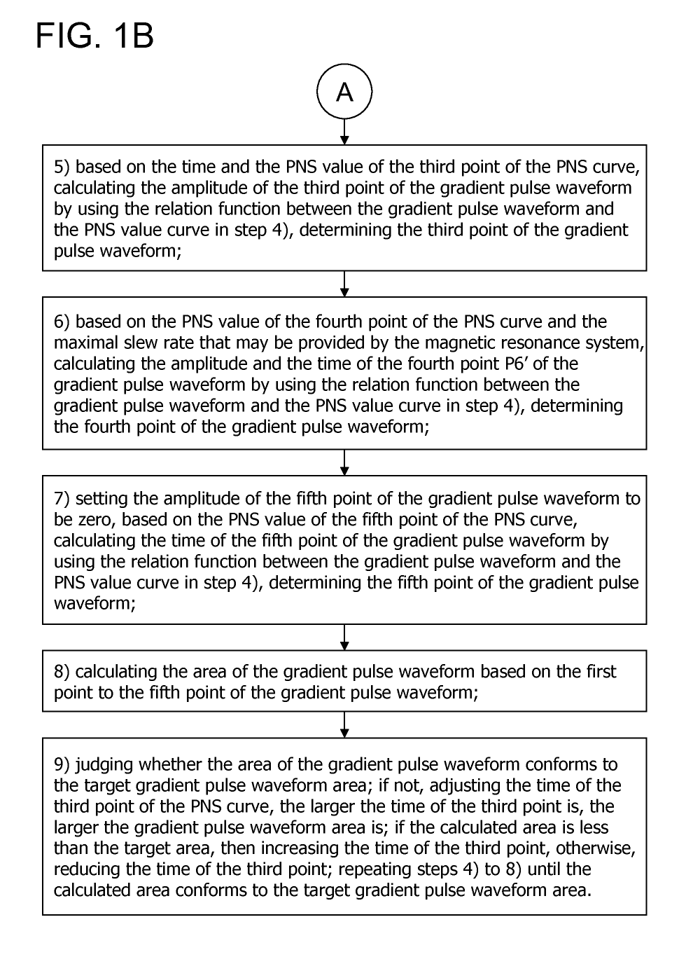

Please see FIGS. 1A and 1B, which show a flow diagram of an embodiment of a method for designing MRI gradient pulse waveform of the present invention, comprising the following steps:

1) Acquiring a slew rate that may be provided by a magnetic resonance system.

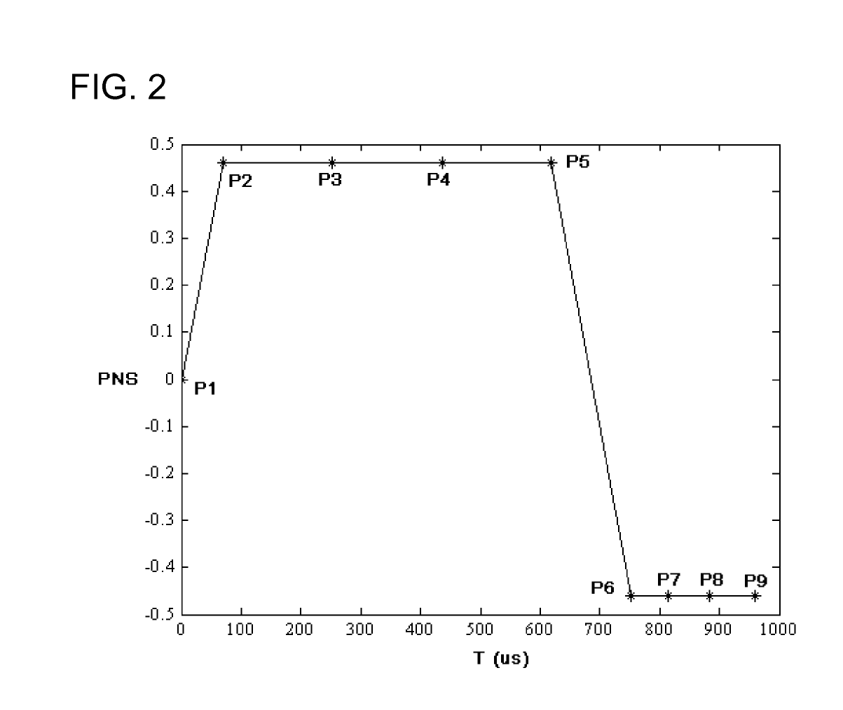

2) Defining target PNS curve, said target PNS curve meets the following requirements, as shown in FIG. 2:

a) both the time and the PNS value of the start point P1 are zero, reaching a maximal PNS value from the start point with the acquired slew rate, acquiring the second point of the PNS curve, the time of the second point of the PNS curve being undetermined; e.g. from point P1 to point P2 in the figure, said acquired slew rate is the maximal slew rate of the system in this embodiment;

b) setting a time randomly, keeping PNS value at a maximal absolute value in the period of time, acquiring the third point of the PNS curve, e.g. point P5 in the figure; of course, PNS value may be kept within a client-set range in the period of time;

c) controlling PNS value to decrease from the positive maximal value to a negative maximal point from the third point, i.e., point P5, with the acquired slew rate, acquiring the fourth point of the PNS curve, the time of the fourth point of the PNS curve being undetermined, e.g. point P6 in the figure; likewise, if coming within a client-set PNS value range, PNS value decreases from the client-set range to a negative maximal value;

d) keeping PNS value at the negative maximal value from the fourth point of the PNS curve, i.e. point P6, to the fifth point of the set PNS curve, e.g., point P9 in the figure, the time of the fifth point of the PNS curve, i.e. point P9, being undetermined; of course, PNS value also may be kept within a client-set range in the period of time.

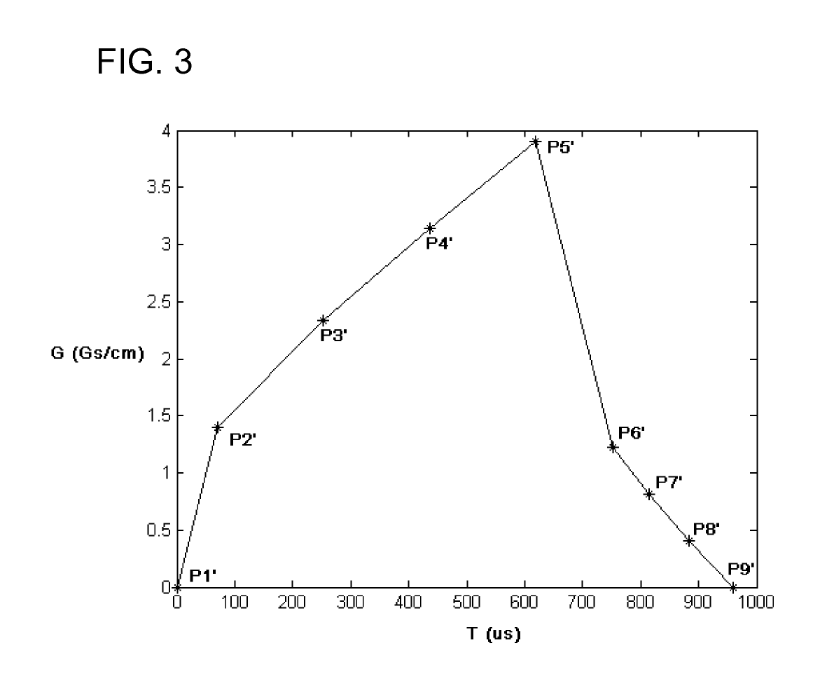

3) Please meanwhile see FIG. 3, setting the start point P1′ of the gradient pulse waveform to be designed; the time and the amplitude thereof are both zero; of course, the time and the amplitude of the start point P r may be random, they may be either set by client or determined by the system.

4) Based on the acquired slew rate and the PNS value of point P2, calculating the amplitude, i.e. the gradient field intensity, and the time of the second point p2′ of the gradient pulse waveform by using the relation function (1) between said gradient pulse waveform and the PNS value curve, the time of the second point P2′ of said gradient pulse waveform being consistent with the time of the second point of the PNS curve, determining the second point P2′ of the gradient pulse waveform based on the time and the amplitude; please meanwhile see FIG. 3.

$${R}_{x} ( t ) = \frac{1}{rb} {\int }_{0}^{t} \frac{{\dot{B}}_{x} ( θ ) c}{{( c + t - θ )}^{2}} d θ \qquad function ( 1 )$$

wherein rb is rheobase, c is chronaxie time, rb and c are both provided by the system; Rx represents PNS value, θ is an intermediate variant from 0˜t; Bₓ represents gradient field intensity, i.e. amplitude; \dot{B}ₓ(θ) represents slew rate, t represents time; when the time is calculated based on the slew rate and the PNS value, the amplitude Bx may be calculated by the calculus dBx/dt of Bₓ.

5) Based on the time and the PNS value of the third point P5 of the PNS curve, calculating the amplitude of the third point P5′ of the gradient pulse waveform by using the relation function (1) between the gradient pulse waveform and the PNS value curve in step 4), the time of the third point P5′ being consistent with the time of the third point P5 of the PNS curve, determining the third point P5′ of the gradient pulse waveform based on the time and the amplitude.

6) Based on the acquired slew rate and the PNS value of the fourth point P6 of the PNS curve, calculating the amplitude and the time of the fourth point P6′ of the gradient pulse waveform by using the relation function (1) between the gradient pulse waveform and the PNS value curve in step 4), the time of the fourth point P6′ of the gradient pulse waveform being consistent with the time of the fourth point P6 of the PNS curve, determining the fourth point P6′ of the gradient pulse waveform based on the time and the amplitude.

7) Setting the amplitude of the fifth point P9′ of the gradient pulse waveform to be zero, based on the PNS value of the fifth point P9 of the PNS curve, calculating the time of the fifth point P9′ of the gradient pulse waveform by using the relation function (1) between the gradient pulse waveform and the PNS value curve in step 4), the time of point P9′ of the gradient pulse waveform being consistent with the time of fifth point P9 of the PNS curve, determining the fifth point P9′ of the gradient pulse waveform based on the time and the amplitude. Of course, the amplitude of the fifth point P9′ of the gradient pulse waveform may be any value set by client.

8) Calculating the area of the gradient pulse waveform based on the first point P1′ to the fifth point P9′ of the gradient pulse waveform.

9) Judging whether the area of the gradient pulse waveform conforms to the area of the target gradient pulse waveform; if not, adjusting the time of the third point P5 of the PNS curve, the larger the time of the third point P5 is, the larger the gradient pulse waveform area is; if the calculated area is less than the target area, then increasing the time of the third point P5, otherwise, reducing the time of the third point P5; repeating steps 4) to 8) until the calculated area conforms to the target gradient pulse waveform area.

Wherein said acquired slew rate is the maximal slew rate of the system in the present embodiment, of course, it may be any value less than the maximal slew rate of the system.

Herein, in order to make simple description, we assume that the target area is large enough and P2′ will not change.

The application of the present invention is illustrated with an example as follows, a gradient pulse waveform is designed according to the following conditions:

Conditions: rb (Rheobase)=23.7 T/s, c (chronaxie)=370 us, effective gradient coil length=34.4 cm, Slew Rate=200 T/m/s, B (amplitude)=5 Gs/cm. Assume that the gradient pulses of the three axes are the same, meanwhile dBx/dt meets the requirement of normal mode of the IEC (International Electrotechnical Commission).

Purpose: PNS=0.8/sqrt(3)=0.4619, gradient pulse waveform area=2000 Gs.us/cm.

The gradient pulse waveform to be designed according to the method stated above is as shown in FIG. 3.

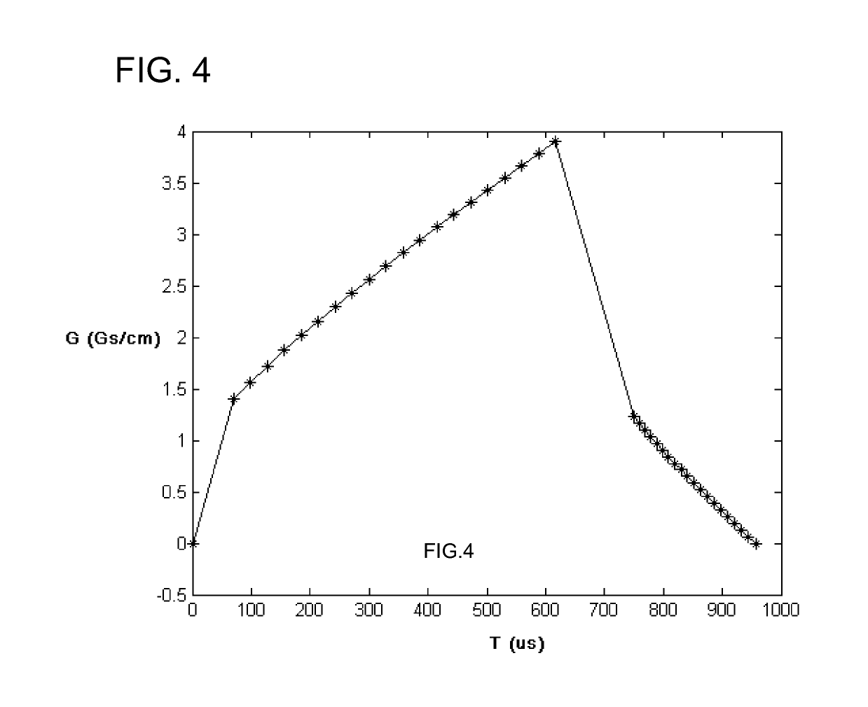

In this example, only 9 corner points are used. In fact, there is no limitation for amount of corner points to get ideal gradient pulse waveform. As shown in FIG. 4, when the number of corner points increases from 9 to 41, the duration of pulse is reduced from 960 us to 957 us, the forms thereof are substantially the same.

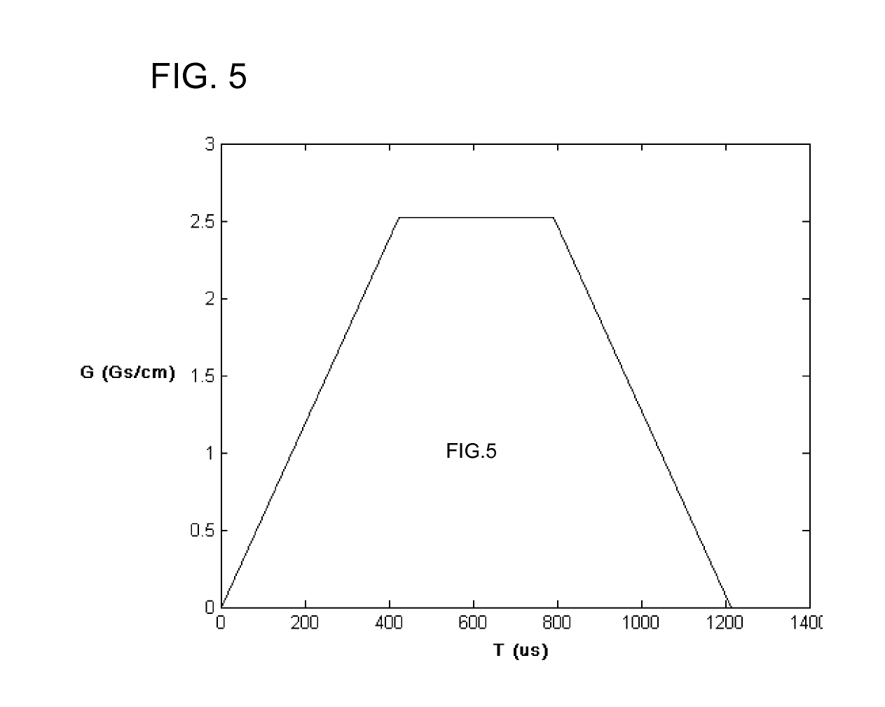

As seen from FIG. 3 and FIG. 5, under the same conditions, the duration of the gradient pulse waveform designed by employing the method of the present invention is only 960 us, while the duration of the gradient pulse waveform in the prior art is 1216 us, the duration reduces by about 20%.

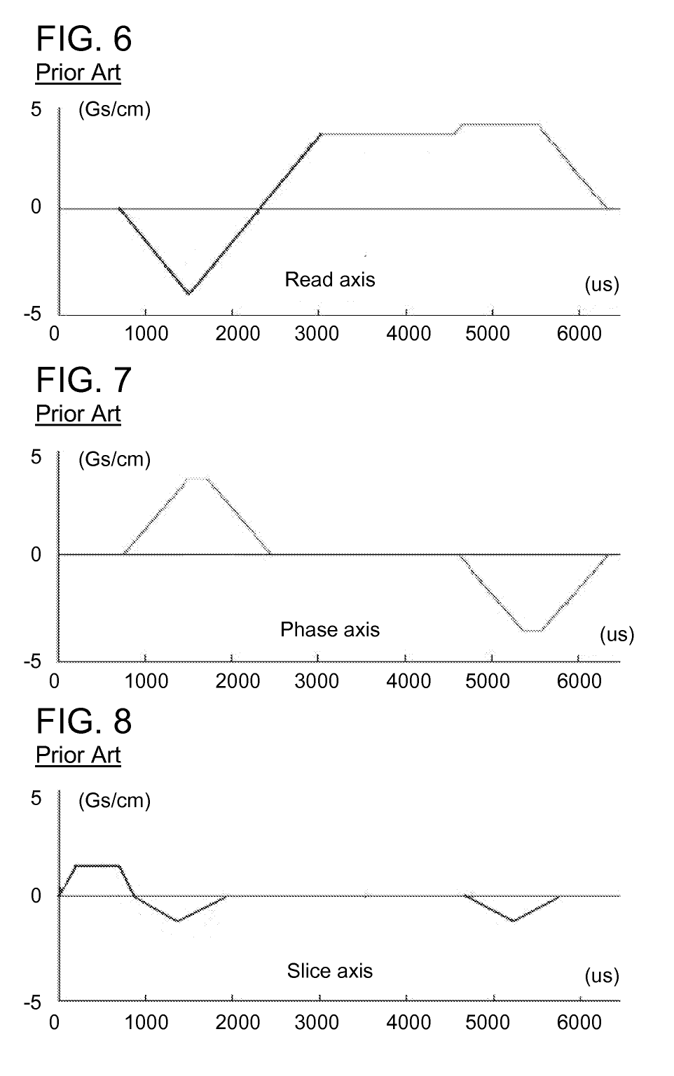

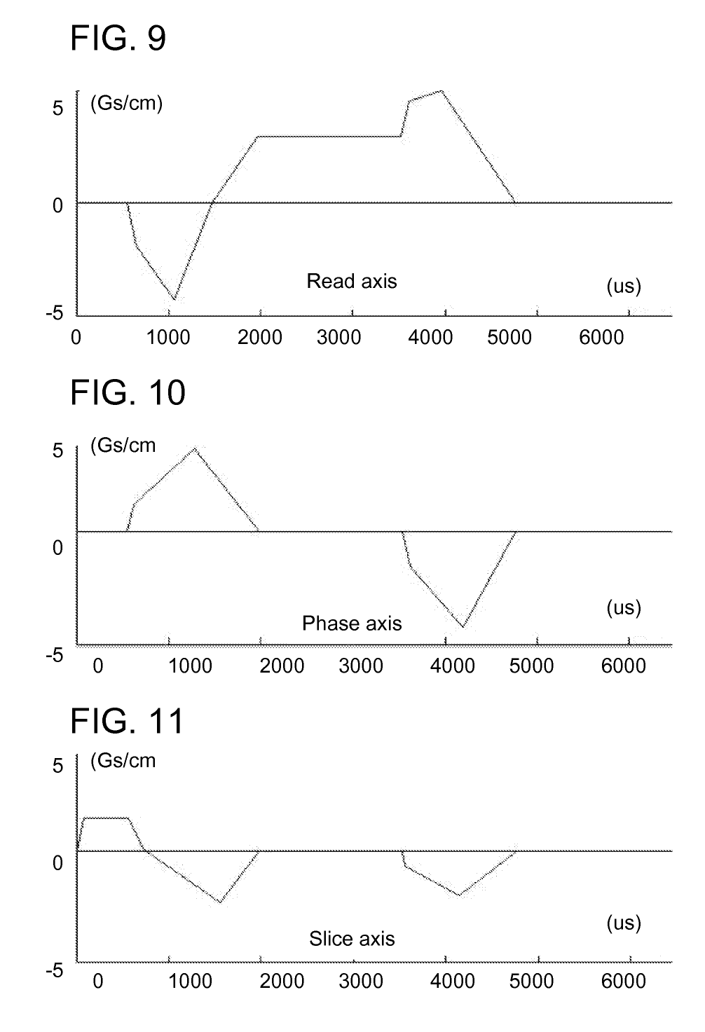

FIGS. 6-8 are schematic diagrams of a gradient pulse waveform designed by using the prior art. Specifically, FIG. 6 is a schematic diagram of a prior art gradient pulse waveform along a Read axis, FIG. 7 is a schematic diagram of the prior art gradient pulse waveform along a Phase axis, and FIG. 8 is a schematic diagram of the prior art gradient pulse waveform along a Slice axis. It can be seen from the figure that the repetition time is 6.2 ms, and the echo time is 2.7 ms. FIGS. 9-11 are schematic diagrams of a gradient pulse waveform designed by using the present invention under condition equal to that of FIGS. 6-8. Specifically, FIG. 9 is a schematic diagram of a gradient pulse waveform along a Read axis, FIG. 10 is a schematic diagram of the gradient pulse waveform along a Phase axis, and FIG. 11 is a schematic diagram of the gradient pulse waveform along a Slice axis. It can be seen from the figure that the time of repetition is 4.9 ms, and the time of echo is 2.04 ms.

The present invention not only is adapted for single pulse, but also can be used at any rising/drop period where gradient field intensity changes.

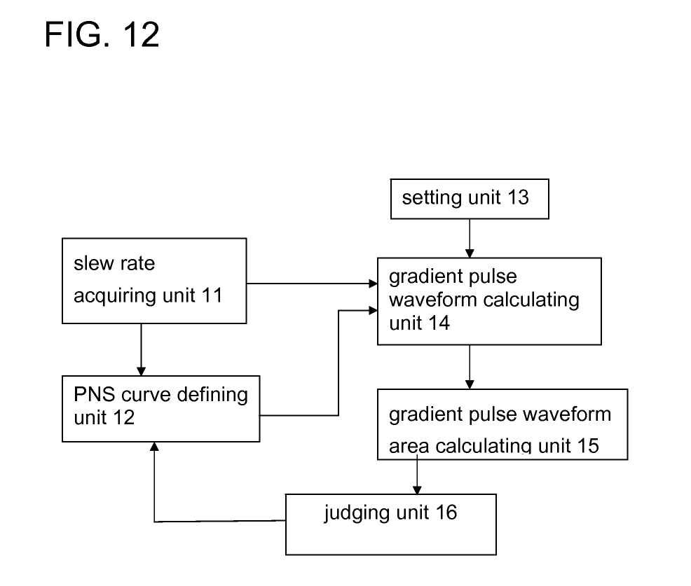

As shown in FIG. 12, an apparatus for designing MRI gradient pulse waveform provided by the present invention, comprising: a slew rate acquiring unit 11, a PNS curve defining unit 12, a setting unit 13, a gradient pulse waveform calculating unit 14, a gradient pulse waveform area calculating unit 15 and a judging unit 16.

Wherein said slew rate acquiring unit 11 is configured to acquire a slew rate that may be provided by a magnetic resonance system.

The PNS curve defining unit 12 is configured to define any target PNS curve, in said implementation form, the PNS curve defined by the PNS curve defining unit 12 satisfies the following conditions (please meanwhile see FIG. 2): a) reaching a maximal PNS value from the start point P1 with the acquired slew rate, acquiring the second point P2 of the PNS curve, the time of the second point P2 of the PNS curve being undetermined, e.g. from point P1 to point P2 in the figure; the time and the PNS value of the start point P1 are zero; b) setting a time randomly, keeping PNS value at a maximal absolute value in the period of time, whereby determining the third point of the PNS curve, e.g. point P5 in the figure; of course, PNS value may be kept within a client-set range in the period of time; c) controlling PNS value to decrease from the positive maximal value to a negative maximal point from point P5 with the acquired slew rate, acquiring the fourth point of the PNS curve, the time of the fourth point of the PNS curve being undetermined, e.g. point P6 in the figure; if coming within a client-set PNS value range, PNS value decreases from the client-set range to a negative maximal value; d) keeping PNS value at the negative maximal value from the fourth point of the PNS curve, i.e. point P6, to the fifth point of the set PNS curve, e.g., point P9 in the figure, the time of the fifth point of the PNS curve, i.e. point P9, being undetermined; of course, the PNS value also may be kept within a client-set range in the period of time.

The setting unit 13 is configured to set the start point P1′ of the gradient pulse waveform to be designed, and sets both the time and the amplitude thereof to be zero. Of course, the time and the amplitude of the start point P1′ may be any, they may be either set by client or determined by the system.

The gradient pulse waveform calculating unit 14 is configured to determine the second point, the third point, the fourth point and the fifth point of the gradient pulse waveform through calculation. The gradient pulse waveform calculating unit 14 calculates the second point of the gradient pulse waveform in the following way: based on the acquired slew rate and the PNS value of the second point P2, calculating the amplitude and the time of the second point P2′ of the gradient pulse waveform by using the relation function (1) between the gradient pulse waveform and the PNS value curve, the time of the second point P2′ of the gradient pulse waveform being consistent with the time of the second point of the PNS curve, determining the second point P2′ of the gradient pulse waveform based on the time and the amplitude. Please meanwhile see FIG. 3.

$${R}_{x} ( t ) = \frac{1}{rb} {\int }_{0}^{t} \frac{{\dot{B}}_{x} ( θ ) c}{{( c + t - θ )}^{2}} d θ \qquad function ( 1 )$$

wherein rb is rheobase, c is chronaxie time; Rx represents PNS value, θ is an intermediate variant from 0˜t; Bₓ represents gradient field intensity, i.e. amplitude; \dot{B}ₓ(θ) represents slew rate, t represents time; after the time is calculated based on the slew rate and the PNS value, the amplitude Bₓ may be calculated by the calculus dBx/dt of Bₓ.

The gradient pulse waveform calculating unit 14 is configured to calculate the third point of the gradient pulse waveform in the following way: based on the time and the PNS value of the third point P5 of the PNS curve, calculating the amplitude of the third point P5′ of the gradient pulse waveform by using the relation function (1) between the gradient pulse waveform and the PNS value curve, the time of the third point P5′ of the gradient pulse waveform being consistent with the time of the third point P5 of the PNS curve, determining the third point P5′ of the gradient pulse waveform based on the time and the amplitude.

The gradient pulse waveform calculating unit 14 is configured to calculate the fourth point of the gradient pulse waveform in the following way: based on the acquired slew rate and the PNS value of the fourth point P6 of the PNS curve, calculating the amplitude and the time of the fourth point P6′ of the gradient pulse waveform by using the relation function (1) between the gradient pulse waveform and the PNS value curve, the time of the fourth point P6′ of the gradient pulse waveform being consistent with the time of the fourth point P6 of the PNS curve, determining the fourth point P6′ of the gradient pulse waveform based on the time and the amplitude.

The gradient pulse waveform calculating unit is configured to calculate the fifth point of the gradient pulse waveform in the following way: setting the amplitude of the fifth point P9′ of the gradient pulse waveform to be zero, based on the PNS value of the fifth point P9 of the PNS curve and the relation function (1) between the gradient pulse waveform and the PNS value curve, calculating the time of the fifth point P9′ of the gradient pulse waveform, determining the fifth point P9′ of the gradient pulse waveform based on the time and the amplitude. The time of the fifth point P9′ of the gradient pulse waveform being consistent with the time of the fifth point P9 of the PNS curve. Of course, the amplitude of the fifth point P9′ of the gradient pulse waveform may be any value set by client.

The gradient pulse waveform area calculating unit 15 is configured to calculate the gradient pulse waveform area based on the first point P1′ to the fifth point P9′ of the gradient pulse waveform.

The judging unit 16 is configured to judge whether the gradient pulse waveform area conforms to the target gradient pulse waveform area based on the area calculated by the gradient pulse waveform area calculating unit; if not, it feeds back information to the PNS curve defining unit. The PNS curve defining unit adjusts the time of the third point P5 of the PNS curve, the larger the time of the third point P5 is, the larger the gradient pulse waveform area is; if the calculated area is less than the target area, then increasing the time of the third point P5, otherwise, reducing the time of the third point P5; until the calculated area conforms to the target gradient pulse waveform area.

Wherein the slew rate acquired by the slew rate acquiring unit 11 is provided by the system, it is the maximal slew rate of the system in the present embodiment, of course, it also may be any value less than the maximal slew rate.

1. A method for designing MRI gradient pulse waveform, said method comprising:
defining a target peripheral nerve stimulation (PNS) curve;
calculating a gradient pulse waveform using a relation function between the gradient pulse waveform and a PNS value curve based on the target PNS curve;
$${R}_{x} ( t ) = \frac{1}{rb} {\int }_{0}^{t} \frac{{\dot{B}}_{x} ( θ ) c}{{( c + t - θ )}^{2}} d θ$$
wherein rb is rheobase, c is chronaxie time, rb and c are both provided by system; Rx represents PNS value, θ is an intermediate variant from 0˜t; Bₓ represents gradient field intensity, i.e. amplitude; \dot{B}ₓ(θ) represents slew rate, t represents time.

2. A method for designing MRI gradient pulse waveform as stated in claim 1, wherein defining the target PNS curve comprises:
acquiring a slew rate from a magnetic resonance system;
defining target peripheral nerve stimulation (PNS) curve based on the acquired slew rate;
reaching a maximal PNS value from a start point with the acquired slew rate, determining a second point of the PNS curve, the time of the second point of the PNS curve being undetermined;
setting a time randomly, from the second point of the PNS curve as a start, keeping PNS value within a client-set range in the period of time, determining a third point of the PNS curve;
controlling PNS to decrease from the client-set range to a negative maximal point from the third point with the acquired slew rate, determining a fourth point of the PNS curve, the time of the fourth point of the PNS curve being undetermined; and
keeping PNS value within a client-set range from the fourth point of the PNS curve to a fifth point of the PNS curve, the time of the fifth point of the PNS curve being undetermined.

3. A method for designing MRI gradient pulse waveform as stated in claim 2, wherein calculating a gradient pulse waveform comprises:
setting the start point of the gradient pulse waveform to be designed;
based on the acquired slew rate and the PNS value of the second point of the PNS curve, calculating the time and the amplitude of the second point of the gradient pulse waveform by using the relation function between the gradient pulse waveform and the PNS value curve, the time of the second point of the gradient pulse waveform being consistent with the time of the second point of the PNS curve, whereby determining the second point of the gradient pulse waveform;
based on the time and the PNS value of the third point of the PNS curve, calculating the amplitude of the third point of the gradient pulse waveform by using the relation function between the gradient pulse waveform and the PNS value curve, the time of the third point of the gradient pulse waveform being consistent with the time of the third point of the PNS curve, whereby determining the third point of the gradient pulse waveform;
based on the acquired slew rate and the PNS value of the fourth point of the PNS curve, calculating the amplitude and the time of the fourth point of the gradient pulse waveform by using the relation function between the gradient pulse waveform and the PNS value curve, the time of the fourth point of the gradient pulse waveform being consistent with the time of the fourth point of the PNS curve, whereby determining the fourth point of the gradient pulse waveform;
based on the amplitude of the fifth point of the gradient pulse waveform provided by client or system, and based on the PNS value of the fifth point of the PNS curve, calculating the time of the fifth point of the gradient pulse waveform by using the relation function between the gradient pulse waveform and the PNS value curve, whereby determining the fifth point of the gradient pulse waveform.

4. A method for designing MRI gradient pulse waveform as stated in claim 3, further comprising calculating the area of the gradient pulse waveform based on the first point to the fifth point of the gradient pulse waveform.

5. A method for designing MRI gradient pulse waveform as stated in claim 4, further comprising:
judging whether the area of the gradient pulse waveform conforms to the target gradient pulse waveform area;
if not, adjusting the time of the third point of the PNS curve, the larger the time of the third point of the PNS curve is, the larger the gradient pulse waveform area is;
if the calculated area is less than the target area, then increasing the time of the third point of the PNS curve, otherwise, reducing the time of the third point of the PNS curve; and
repeating the calculations to determine the time and amplitude of the second point, the time and amplitude of the third point, the time and amplitude of the fourth point, the time of the fifth point, and the area of the gradient pulse waveform until the calculated area conforms to the target gradient pulse waveform area.

6. A method for designing MRI gradient pulse waveform as stated in claim 2, wherein, when the time and the PNS value of the start point of the target PNS curve are zero, the acquired slew rate is the maximal slew rate of system.

7. A method for designing MRI gradient pulse waveform as stated in claim 2, wherein from the second point of the PNS curve as a start, the PNS value is kept at a positive maximal value within the period of time, and the PNS value is kept at a negative maximal value from the fourth point of the PNS curve to the fifth point of the PNS curve.

8. A method for designing MRI gradient pulse waveform as stated in claim 3, wherein when the time and the amplitude of the start point of the gradient pulse waveform are zero, the amplitude of the fifth point of the gradient pulse waveform is zero.

9. A method for designing MRI gradient pulse waveform as stated in claim 3, wherein the time of the fifth point of the gradient pulse waveform is consistent with the time of the fifth point of the PNS curve.

10. An apparatus for use in designing an MRI gradient pulse waveform, said apparatus comprising:
a peripheral nerve stimulation (PNS) curve defining unit configured to define a target PNS curve; and
a gradient pulse waveform calculating unit configured to:
determine a gradient pulse waveform to be designed through calculation;
calculate the gradient pulse waveform by using a relation function between the gradient pulse waveform and a PNS value curve based on the target PNS curve;
$${R}_{x} ( t ) = \frac{1}{rb} {\int }_{0}^{t} \frac{{\dot{B}}_{x} ( θ ) c}{{( c + t - θ )}^{2}} d θ$$
wherein rb is rheobase, c is chronaxie time, rb and c are both provided by the system; Rx represents PNS value, θ is an intermediate variant from 0˜t; Bₓ represents gradient field intensity, i.e. amplitude; \dot{B}ₓ(θ) represents slew rate, t represents time.

11. An apparatus for designing MRI gradient pulse waveform as stated in claim 10, further comprising a slew rate acquiring unit configured to acquire a slew rate that may be provided by a magnetic resonance said PNS curve defining unit is configured to define the PNS curve such that the PNS curve by:
reaching a maximal PNS value from the start point with the acquired slew rate, determining the second point of the PNS curve, the time of the second point of the PNS curve being undetermined;
setting a time randomly, from the second point of the PNS curve as a start, keeping PNS value within a client-set range in the period of time, determining the third point of the PNS curve;
controlling PNS to decrease from the client-set range to a negative maximal point from the third point with the acquired slew rate, determining the fourth point of the PNS curve, the time of the fourth point of the PNS curve being undetermined;
keeping PNS value within a client-set range from the fourth point of the PNS curve to the fifth point of the PNS curve, the time of the fifth point of the PNS curve being undetermined.

12. An apparatus for designing MRI gradient pulse waveform as stated in claim 11, further comprising a setting unit configured to set the start point of the gradient pulse waveform to be designed, said gradient pulse waveform calculating unit is configured to determine the second point, the third point, the fourth point and the fifth point of the gradient pulse waveform to be designed through calculation.

13. An apparatus for designing MRI gradient pulse waveform as stated in claim 12, wherein said gradient pulse waveform calculating unit is configured to:
calculate the second point of the gradient pulse waveform by the following way: based on the acquired slew rate and the PNS value of the second point of the PNS curve, calculating the amplitude and the time of the second point of the gradient pulse waveform by using the relation function between the gradient pulse waveform and the PNS value curve, the time of the second point of the gradient pulse waveform being consistent with the time of the second point of the PNS curve, determining the second point of the gradient pulse waveform based on the time and the amplitude;
calculate the third point of the gradient pulse waveform by the following way: based on the time and the PNS value of the third point of the PNS curve, calculating the amplitude of the third point of the gradient pulse waveform by using the relation function between the gradient pulse waveform and the PNS value curve, the time of the third point of the gradient pulse waveform being consistent with the time of the third point of the PNS curve, determining the third point of the gradient pulse waveform based on the time and the amplitude;
calculate the fourth point of the gradient pulse waveform by the following way: based on the acquired slew rate and the PNS value of the fourth point of the PNS curve, calculating the amplitude and the time of the fourth point of the gradient pulse waveform by using the relation function between the gradient pulse waveform and the PNS value curve, the time of the fourth point of the gradient pulse waveform being consistent with the time of the fourth point of the PNS curve, determining the fourth point of the gradient pulse waveform based on the time and the amplitude;
calculate the fifth point of the gradient pulse waveform by the following way: based on the amplitude of the fifth point of the gradient pulse waveform set by client or system, and based on the PNS value of the fifth point of the PNS curve, calculating the time of the fifth point of the gradient pulse waveform by using the relation function between the gradient pulse waveform and the PNS value curve, determining the fifth point of the gradient pulse waveform based on the time and the amplitude, the time of the fifth point of said gradient pulse waveform being consistent with the time of the fifth point of the PNS curve.

14. An apparatus for designing MRI gradient pulse waveform as stated in claim 13, further comprising a gradient pulse waveform area calculating unit configured to calculate the gradient pulse waveform area based on the first point to the fifth point of the gradient pulse waveform.

15. An apparatus for designing MRI gradient pulse waveform as stated in claim 14, further comprising a judging unit configured to:
judge whether the gradient pulse waveform area conforms to the target gradient pulse waveform area based on the area calculated by the gradient pulse waveform area calculating unit;
if not, it feeds back information to the PNS curve defining unit, such that said PNS curve defining unit adjusts the time of the third point of the PNS curve based on the feedback of the judging unit, the larger the time of the third point is, the larger the gradient pulse waveform area is;
if the calculated area is less than the target area, then increasing the time of the third point; otherwise, reducing the time of the third point; until the calculated area conforms to the target gradient pulse waveform area.

16. An apparatus for designing MRI gradient pulse waveform as stated in claim 11, wherein when the time and the PNS value of the start point of the target PNS curve are zero, the acquired slew rate is the maximal slew rate of system.

17. An apparatus for designing MRI gradient pulse waveform as stated in claim 11, wherein when the PNS value is kept at a positive maximal value from the second point of the PNS curve as a start, the PNS value is kept at a negative maximal value from the fourth point of the PNS curve to the fifth point of the PNS curve.

18. An apparatus for designing MRI gradient pulse waveform as stated in claim 13, characterized in that, wherein when the time and the amplitude of the start point of the gradient pulse waveform are zero, the amplitude of the fifth point of the gradient pulse waveform is zero.

19. A magnetic resonance imaging system comprising:
a slew rate acquisition unit; and
an apparatus coupled to said slew rate acquisition unit and configured to design an MRI gradient pulse waveform, said apparatus comprising:
a peripheral nerve stimulation (PNS) curve defining unit configured to define a target PNS curve; and
a gradient pulse waveform calculating unit configured to:
determine a gradient pulse waveform to be designed through calculation;
calculate the gradient pulse waveform by using a relation function between the gradient pulse waveform and a PNS value curve based on the target PNS curve and based on a rheobase, a chronaxie time, the PNS value curve, an intermediate variant between a time zero and a time t, and a gradient field intensity.

20. A magnetic resonance imaging system in accordance with claim 19, further comprising a slew rate acquiring unit configured to acquire a slew rate that may be provided by a magnetic resonance said PNS curve defining unit is configured to define the PNS curve such that the PNS curve by:
reaching a maximal PNS value from the start point with the acquired slew rate, determining the second point of the PNS curve, the time of the second point of the PNS curve being undetermined;
setting a time randomly, from the second point of the PNS curve as a start, keeping PNS value within a client-set range in the period of time, determining the third point of the PNS curve;
controlling PNS to decrease from the client-set range to a negative maximal point from the third point with the acquired slew rate, determining the fourth point of the PNS curve, the time of the fourth point of the PNS curve being undetermined;
keeping PNS value within a client-set range from the fourth point of the PNS curve to the fifth point of the PNS curve, the time of the fifth point of the PNS curve being undetermined.
